(19) United States
(12) Patent Application Publication
(10) Pub. No.: US 2010/0069609 A1
(43) Pub. Date: Mar. 18, 2010

(54) CHEMICAL METHODS AND APPARATUS

(76) Inventor: Erik Arstad, London (GB)

Correspondence Address: GE HEALTHCARE, INC., IP DEPARTMENT 101 CARNEGIE CENTER, PRINCETON, NJ, 08540-6231, US

(21) Appl. No.: 12/305,382

(22) PCT Filed: Jun. 20, 2007
(86) PCT No.: PCT/GB07/02283

§ 371 Date: Jun. 16, 2009

Related U.S. Application Data
(60) Provisional application No. 60/805,373, filed on Jun. 21, 2006.
(60) Provisional application No. 60/867,612, filed on Nov. 29, 2006.

(51) Int. Cl. C07K 1/13; B01J 19/00

(52) U.S. Cl. 530/345; 422/211

(57) ABSTRACT

The invention relates to methods and apparatus for labelling a biologically active vector such as a peptide with reporter moiety such as a radionuclide. The methods comprise reaction of a compound of formula (I) with a compound of formula (II): R*-L2-N3 (II) or, a compound of formula (III) with a compound of formula (IV) wherein: L1, L2, L3, and L4 are each Linker groups; R* is a reporter moiety; in a narrow bore copper vessel. Microfluidic devices for performing the methods of the invention are also claimed.

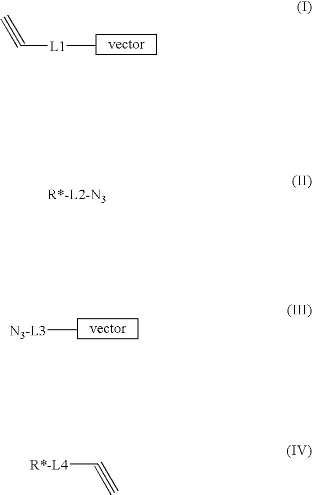

The invention relates to methods and apparatus for labelling a biologically active vector such as a peptide with reporter moiety such as a radionuclide. The resultant labelled conjugates are useful as diagnostic agents, for example, as radiopharmaceuticals more specifically for use in Positron Emission Tomography (PET) or Single Photon Emission Computed Tomography (SPECT) or for radiotherapy.

The application of radiolabelled bioactive peptides for diagnostic imaging is gaining importance in nuclear medicine. Biologically active molecules which selectively interact with specific cell types are useful for the delivery of radioactivity to target tissues. For example, radiolabelled peptides have significant potential for the delivery of radionuclides to tumours, infarcts, and infected tissues for diagnostic imaging and radiotherapy. ¹⁸F, with its half-life of approximately 110 minutes, is the positron-emitting nuclide of choice for many receptor imaging studies. Therefore, ¹⁸F-labelled bioactive peptides have great clinical potential because of their utility in PET to quantitatively detect and characterise a wide variety of diseases. Other useful radionuclides include ¹¹C, radioiodine such as ¹²⁵I, ¹²³I, ¹²⁴I, ¹³¹I and ^{99m}Tc.

To date, a lack of rapid and generally applicable methods for peptide and biomolecule labelling has hampered the use of peptides and biomolecules as diagnostic agents. Therefore, there still exists a need for labelling agents such as ¹⁸F-labelled prosthetic groups and methodologies, which allow rapid, chemoselective introduction of a label such as a radionuclide, for example ¹⁸F, particularly into peptides, under mild conditions to give labelled products in high radiochemical yield and purity. Co-pending application WO2006/067376 describes methods and reagents for labelling a vector such as a peptide for diagnostic imaging. Additionally, there is a need for such methodologies which are amenable to automation to facilitate preparation of diagnostic agents in the clinical setting. The inventors have now found that the methods of WO2006/067376 can be improved by performing the method in a narrow bore copper vessel which serves as a catalyst as well as reaction vessel. In this way, the radiochemical yield can be increased from around 86% to over 99%. In addition, the narrow bore copper vessel may be readily incorporated in an automated synthesis system.

The present invention provides a method for labelling a vector comprising reaction of a compound of formula (I) with a compound of formula (II):

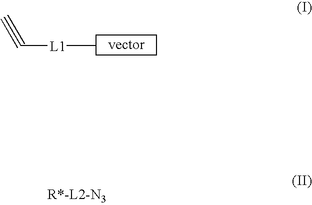

or,
a compound of formula (III) with a compound of formula (IV)

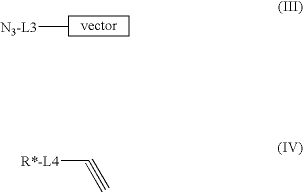

wherein:
L1, L2, L3, and L4 are each Linker groups;
R* is a reporter moiety;
to give a conjugate of formula (V) or (VI) respectively:

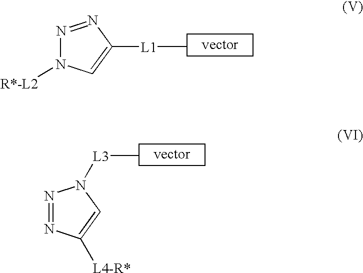

wherein L1, L2, L3, L4, and R* are as defined above;
characterised in that the reaction is performed in a narrow bore copper vessel.

The Linker groups L1, L2, L3, and L4 are each independently a C₁₋₆₀ hydrocarbyl group, suitably a C₁₋₃₀ hydrocarbyl group, optionally including 1 to 30 heteroatoms, suitably 1 to 10 heteroatoms such as oxygen or nitrogen. Suitable Linker groups include alkyl, alkenyl, alkynyl chains, aromatic, polyaromatic, and heteroaromatic rings any of which may be optionally substituted for example with one or more ether, thiooether, sulphonamide, or amide functionality, monomers and polymers comprising ethyleneglycol, amino acid, or carbohydrate subunits.

The term “hydrocarbyl group” means an organic substituent consisting of carbon and hydrogen, such groups may include saturated, unsaturated, or aromatic portions.

The Linker groups L1, L2, L3, and L4 may be chosen to provide good in vivo pharmacokinetics, such as favourable excretion characteristics in the resultant compound of formula (V) or (VI). The use of linker groups with different lipophilicities and or charge can significantly change the in vivo pharmacokinetics of the peptide to suit the diagnostic need. For example, where it is desirable for a compound of formula (V) or (VI) to be cleared from the body by renal excretion, a hydrophilic linker is used, and where it is desirable for clearance to be by hepatobiliary excretion a hydrophobic linker is used. Linkers including a polyethylene glycol moiety have been found to slow blood clearance which is desirable in some circumstances.

R* is a reporter moiety which is detectable by any imaging modality, such as a reporter suitable for in vivo optical imaging, a reporter comprising a radionuclide, or a reporter comprising an isotope suitable for use in Magnetic Resonance Imaging (MRI) or Magnetic Resonance Spectroscopy (MRS). R* preferably comprises a radionuclide for example a positron-emitting radionuclide. Suitable positron-emitting radionuclides for this purpose include ¹¹C, ¹⁸F, ⁷⁵Br, ⁷⁶Br, ¹²⁴I, ⁸²Rb, ⁶⁸Ga, ⁶⁴Cu and ⁶²Cu, of which ¹¹C and ¹⁸F are preferred. In one aspect of the invention, the radionuclide is ¹⁸F. Other useful radionuclides include ¹²³I, ¹²⁵I, ¹³¹I, ²¹¹At, ^{99m}Tc, and ¹¹¹In. Metallic radionuclides are suitably incorporated into a chelating agent, for example by direct incorporation by methods known to the person skilled in the art. Chelation of a metallic reporter is preferably performed prior to reaction of the compound of formula (I) or (IV) with a compound of formula (II) or (III) respectively, to avoid chelation of the Cu(I) catalyst.

Suitable chelating agents comprised in R*, include those of Formula X

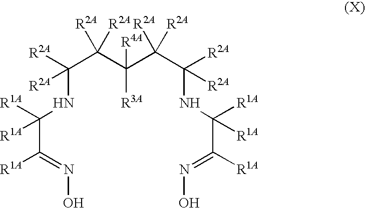

where:
each R^{1A}, R^{2A}, R^{3A} and R^{4A} is independently an R^{A} group;
each R^{A} group is independently H or C₁₋₁₀ alkyl, C₃₋₁₀ alkylaryl, C₂₋₁₀ alkoxyalkyl, C₁₋₁₀ hydroxyalkyl, C₁₋₁₀ alkylamine, C₁₋₁₀ fluoroalkyl, or 2 or more R^{A} groups, together with the atoms to which they are attached form a carbocyclic, heterocyclic, saturated or unsaturated ring,
or R* can comprise a chelating agent given by formula (I), (ii), (iii), or (iv)

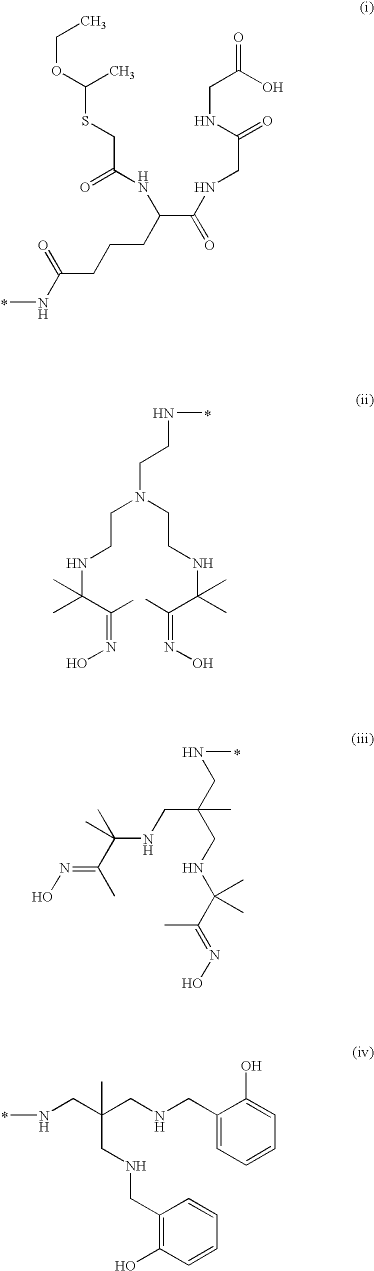

A preferred example of a chelating agent is represented by formula (v).

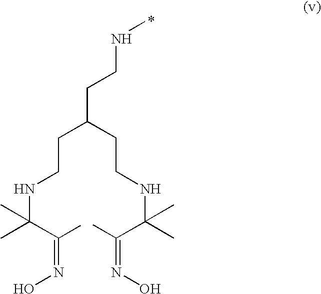

Compounds of formula (II) or (IV) comprising chelating agents of Formula X can be radiolabelled to give good radiochemical purity (RCP), at room temperature, under aqueous conditions at near neutral pH.

When R* is a reporter suitable for in vivo optical imaging, the reporter is any moiety capable of detection either directly or indirectly in an optical imaging procedure. The reporter may be a light scatterer (e.g. a coloured or uncoloured particle), a light absorber or a light emitter. More preferably the reporter is a dye such as a chromophore or a fluorescent compound. The dye can be any dye that interacts with light in the electromagnetic spectrum with wavelengths from the ultraviolet light to the near infrared. Most preferably the reporter has fluorescent properties. Preferred organic chromophoric and fluorophoric reporters include groups having an extensive delocalized electron system, e.g. cyanines, merocyanines, indocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes. Fluorescent proteins, such as green fluorescent protein (GFP) and modifications of GFP that have different absorption/emission properties are also useful. Complexes of certain rare earth metals (e.g., europium, samarium, terbium or dysprosium) are used in certain contexts, as are fluorescent nanocrystals (quantum dots).

Particular examples of chromophores which may be used include: fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750.

Suitable methods for the introduction of a chromophore are detailed in WO 98/048838.

When R* comprises an isotope suitable for use in Magnetic Resonance Imaging (MRI) or Magnetic Resonance Spectroscopy (MRS), such isotopes are suitably selected from ¹⁹F and ¹³C.

In formulae (I) and (III) and in other aspects of the invention unless specifically stated otherwise, suitable vectors for labelling are peptides, which may include somatostatin analogues, such as octreotide, bombesin, vasoactive intestinal peptide, chemotactic peptide analogues, α-melanocyte stimulating hormone, neurotensin, Arg-Gly-Asp peptide, human pro-insulin connecting peptide, insulin, endothelin, angiotensin, bradykinin, endostatin, angiostatin, glutathione, calcitonin, Magainin I and II, luteinizing hormone releasing hormone, gastrins, cholecystochinin, substance P, vasopressin, formyl-norleucyl-leucyl-phenylalanyl-norleucyl-tyrosyl-lysine, Annexin V analogues, Vasoactive Protein-1 (VAP-1) peptides, and caspase peptide substrates. Preferred peptides for labelling are Arg-Gly-Asp peptide and its analogues, such as those described in WO 01/77415 and WO 03/006491, preferably a peptide comprising the fragment

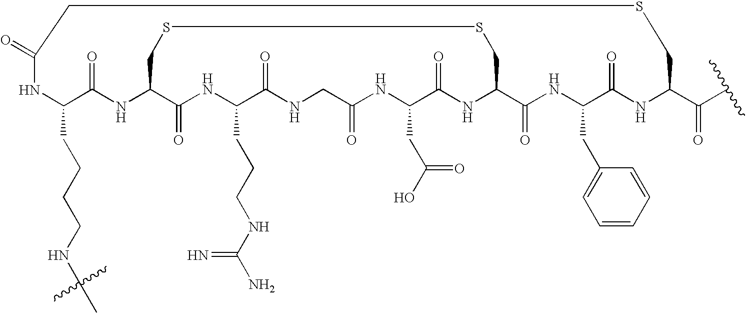

more preferably the peptide of formula (A):

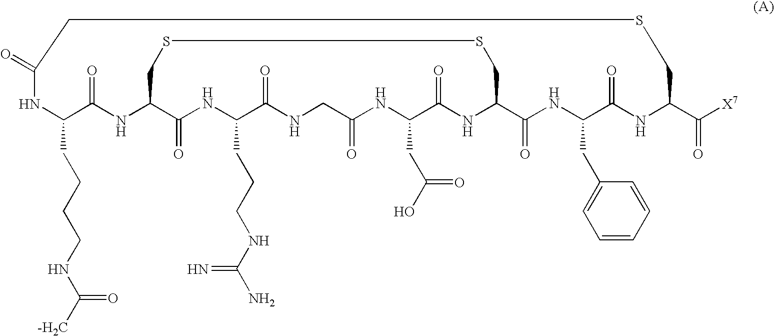

wherein X⁷ is either —NH₂ or

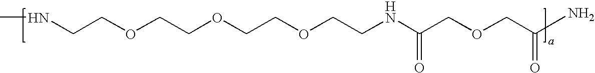

wherein a is an integer of from 1 to 10, preferably a is 1.

As will be appreciated by the skilled person, the methods of the invention may also be used for radiolabelling of other biomolecules such as proteins, hormones, polysaccharides, oligonucleotides, and antibody fragments, cells, bacteria, viruses, as well as small drug-like molecules to provide a variety of diagnostic agents. In formulae (I) and (III) and in other aspects of the invention unless specifically stated otherwise, particularly suitable vectors for radiolabelling are peptides, proteins, hormones, cells, bacteria, viruses, and small drug-like molecules.

The reaction of compound of formula (I) with compound of formula (II) or of compound of formula (III) with compound of formula (IV) in a narrow bore copper vessel, may be effected in a suitable solvent, for example acetonitrile, a C₁₋₄ alkylalcohol, dimethylformamide, tetrahydrofuran, or dimethylsulphoxide, or aqueous mixtures of any thereof, or in water and at a temperature of from 5° C. to 200° C., preferably from 50° C. to 150° C.

The narrow bore copper vessel used to perform the reaction preferably takes the form of a tube with a narrow bore, for example an HPLC loop with no solid support packing. The narrow bore copper vessel is conveniently made from metallic copper, or as would be understood by the person skilled in the art, the narrow bore vessel may be composed of some other suitable material but having an internal surface of metallic copper. The internal diameter of the narrow bore copper vessel is usually in the range of about 1 micrometer to 1.5 mm, preferably 40 to 200 μm. It is particularly convenient if the narrow bore copper vessel is open at both ends so that the reagents can be flushed through.

The length of the narrow bore copper vessel will be chosen such that it is long enough for the reaction to be effected but is sufficiently short to minimise residence time in the vessel. A convenient length for the narrow bore copper vessel when in the form of a tube with a narrow bore, is from about 5 cm to 50 cm long, more usually 5 cm to 20 cm and typically about 15 cm.

During reaction, it is thought that Cu(I) intrinsically presented on the internal surface of the narrow bore copper vessel functions as a catalyst.

In a further aspect of the invention, the narrow bore copper vessel used to perform the reaction is a microfluidic device comprising a device body defining a first apperture, a second apperture, and at least one elongate microfluidic passageway in fluid communication therebetween wherein at least a portion of the microfluidic passageway is defined by a metallic copper portion of the device body. In such microfluidic devices, predetermined microfluidic passageways, typically 10-300 μm, more typically 50-300 μm in diameter, are etched or otherwise machined into a device body, conveniently on a surface thereof. The device body is conveniently formed from a copper block or alternatively is, for example, glass, silicon, polymer, or metal and a copper coating is applied to the microfluidic passageways formed therein by sputtering, electroplating or other deposition technique.

These microfluidic passageways may be partially defined by way of a cover plate, preferably made from copper, or alternatively made from another metal or more commonly glass coated with copper as described above. Defining the microfluidic passageways by way of a cover plate creates a contained network capable of manipulating picolitre volumes of liquid or gas. The method used to seal the cover plate in place depends on the materials selected but is conveniently clamping, optionally including an inert gasket seal (for example a Teflon™ seal) between the two surfaces. The devices can handle flows of up to hundreds of microlitres per minute. This could be increased further, for example, by stacking multiple devices. These devices are designed to be used either with pumps, micro syringe pumps (for example those available from Kloehen Limited, Las Vegas, USA) or under electroosmotic flow using fused silica capillaries for interfacing with reagents. The cover plate optionally defines part of the microfluidic passageway.

In a preferred embodiment, the microfluidic device is formed by etching microfluidic passageways into a copper block, which may be achieved using a chemical echant (for example, ferric chloride) and then covering with a copper cover plate which optionally defines at least part of the microfluidic passageways.

Certain microfluidic devices useful for performing methods of the invention as described above, are novel, and therefore form a further aspect of the invention.

Therefore, according to a further aspect of the invention, there is provided a microfluidic device for performing a method according to the invention characterised in that said device comprises a device body defining a first apperture (c), a second apperture (d), and at least one elongate microfluidic passageway (a) in fluid communication therebetween wherein at least a portion of the microfluidic passageway (a) is defined by a metallic copper portion of the device body. Suitably, the device body defining the at least one microfluidic passageway is formed from metallic copper.

In a further aspect, the device body further comprises a base portion and a cover portion defining said first apperture, second apperture, and at least one elongate microfluidic passageway in fluid communication therebetween. Suitably, the microfluidic passageway comprises a channel formed in either the base portion or the cover portion or in both thereof in overlying registry.

According to a yet further aspect of the invention, there is provided a microfluidic device as described above wherein the microfluidic passageway contains a compound of formula (I), (II), (III), or (IV) as defined hereinbefore.

Use of microfabricated or microfluidic devices for performing radiosynthesis is described in WO03/078358. Reviews of methods for construction of microfabricated devices and their application inter alia in synthetic chemistry, may be found in DeWitt, (1999) “Microreactors for Chemical Synthesis”, Current Opinion in Chemical Biology, 3:350-6; Haswell, Middleton et al (2001) “The Application of Microreactors to Synthetic Chemistry”, Chemical Communications: 391-8; Haswell and Skelton (2000) “Chemical and Biochemical Microreactors”, Trends in Analytical Chemistry 19(6), 389-395; and Jensen (2001) “Microreaction Engineering—Is Small Better?” Chemical Engineering Science, 56:293-303.

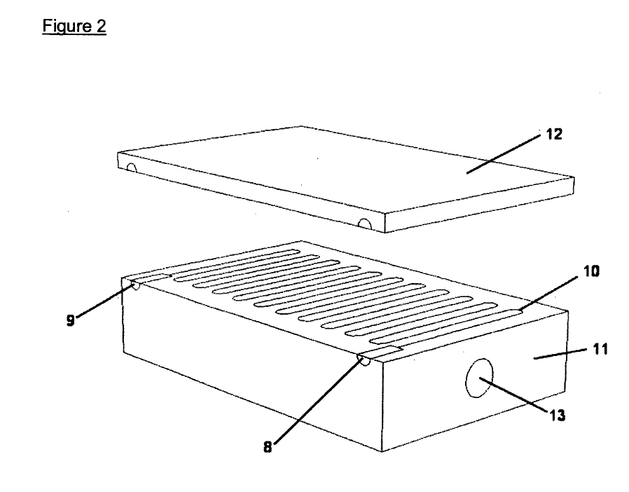

One embodiment of this aspect of the invention is now described with reference to FIG. 2 which provides an exploded view of a microfabricated device according to the invention and suitable for performing a method according to the invention. The copper microfluidic passageway (10), formed in the base portion (11), has a length of 1 metre and an inner diameter of 0.22 mm (tube volume 38 μL). A cover plate (12) is clamped in place to seal the microfluidic passageway. A gas tight syringe (Hamilton, 500 μl) (not shown) is connected to the microfabricated device via a fine bore plastic inlet tube (not shown). The plastic inlet tube is connected to the microfabricated device using a suitable compression fitting via a threaded inlet port (8). A similar method is used to connect an outlet tube via an outlet port (9). A reaction mixture may be pumped through the microfabricated device at temperatures of up to 300° C. and at flowrates of up to 0.5 ml/min. An electric heating cartridge placed in a heating cavity (13) can be heated using a suitable temperature controller.

The present invention provides a chemoselective approach to radiolabelling where the exact site of introduction of the label is pre-selected during the synthesis of the peptide or vector precursor. The ligation reaction occurring at a pre-determined site in the vector gives only one possible product. This methodology is therefore chemoselective, and its application is considered generic for a wide range of peptides, biomolecules and low-molecular weight drugs. Additionally, both alkyne and azide functionalities are stable under most reaction conditions and are unreactive with most common peptide functionalities—thus minimising the protection and deprotection steps required during the labelling synthesis. Furthermore, the triazole ring formed during the labelling reaction does not hydrolise and is highly stable to oxidation and reduction, meaning that the labelled vector has high in vivo stability. The triazole ring is also comparable to an amide in size and polarity such that the labelled peptides or proteins are good mimics for their natural counterparts.

Compounds of formula (I) and (III) wherein the vector is a peptide or protein may be prepared by standard methods of peptide synthesis, for example, solid-phase peptide synthesis, for example, as described in Atherton, E. and Sheppard, R. C.; “Solid Phase Synthesis”; IRL Press: Oxford, 1989. Incorporation of the alkyne or azide group in a compound of formula (I) or (III) may be achieved by reaction of the N or C-terminus of the peptide or with some other functional group contained within the peptide sequence, modification of which does not affect the binding characteristics of the vector. The alkyne or azide groups are preferably introduced to a compound of formula (I) or (III) by formation of a stable amide bond, for example formed by reaction of a peptide amine function with an activated acid or alternatively reaction of a peptide acid function with an amine function and introduced either during or following the peptide synthesis. Methods for incorporation of the alkyne or azide group into vectors such as cells, viruses, bacteria may be found in H. C. Kolb and K. B. Sharpless, Drug Discovery Today, Vol 8 (24), December 2003 and the references therein. Suitable intermediates useful for incorporation of the alkyne or azide group in a compound of formula (I) or (III) include:

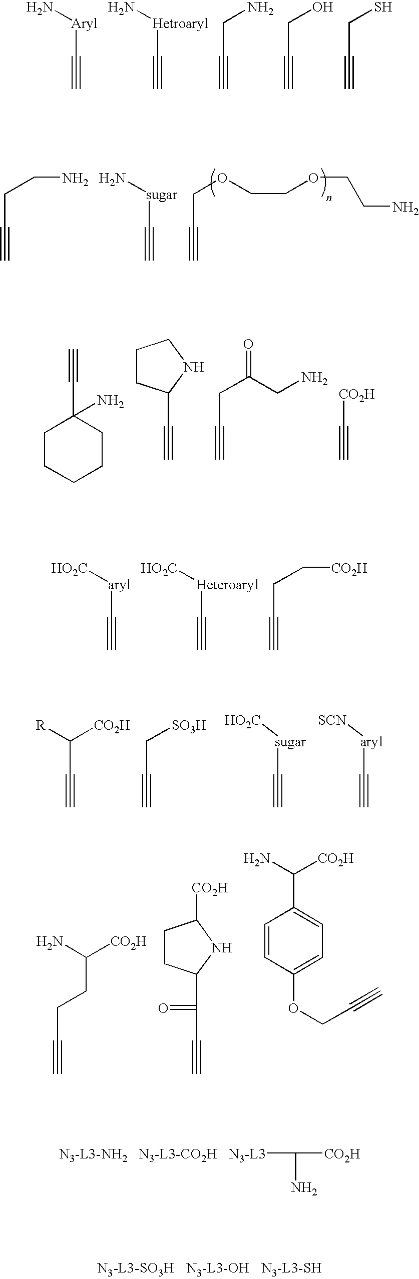

Preferred compounds of formula (IV) for use in the methods of the invention include:

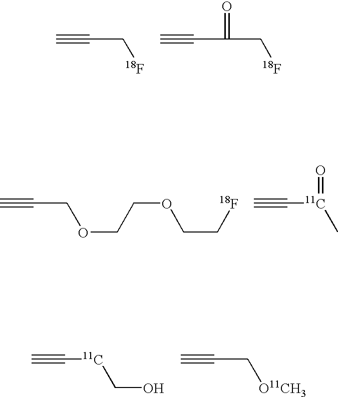

Compounds of formula (II) wherein R* comprises a ¹¹C radiolabel may be prepared for example according to the scheme:

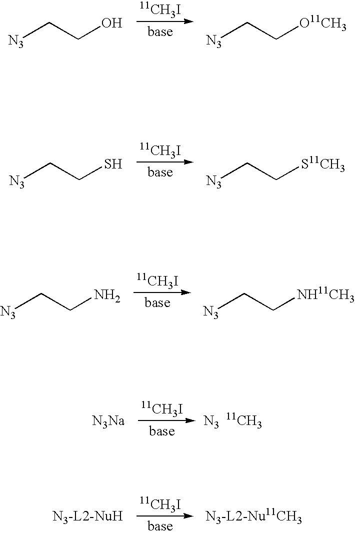

wherein —NuH is a nucleophilic reactive centre such as a hydroxyl, thiol or amine functionality.

Compounds of formula (II) wherein R* is ¹⁸F, may be prepared by either electrophilic or nucleophilic fluorination reactions, for example:

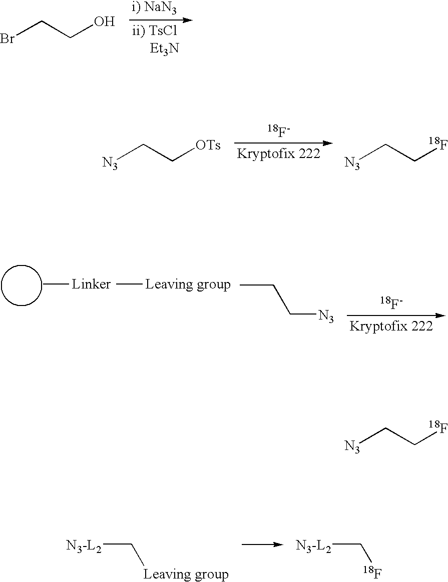

Suitable radiofluorination methods for preparation of a compound of formula (II) include reaction of the precursor incorporating a leaving group (such as an alkyl or aryl sulphonate, for example mesylate, triflate, or tosylate; nitro, or a trialkylammonium salt) with 18F⁻ in the presence of a phase transfer agent such as a cyclic polyether, for example 18-Crown-6 or Kryptofix 2.2.2. This reaction may be performed in solution phase (using an aprotic solvent such as acetonitrile as solvent) under standard conditions known in the art [for example, M. J. Welch and C. S. Redvanly “Handbook of Radiopharmaceuticals”, published by Wiley], or using a solid support to facilitate purification of the compound of formula (II) using the methods described in WO 03/002157.

Compounds of formula (IV) may be prepared from suitable acetylene precursors by methods analogous to those described for synthesis of compounds of formula (II).

The labelled vectors of formulae (V) and (VI) may be administered to patients for in vivo imaging in amounts sufficient to yield the desired signal, typical radionuclide dosages for PET or SPECT imaging of 0.01 to 100 mCi, preferably 0.1 to 50 mCi will normally be sufficient per 70 kg bodyweight.

The labelled vectors of formula (V) or (VI) may therefore be formulated for administration using physiologically acceptable carriers or excipients in a manner fully within the skill of the art. For example, the compounds, optionally with the addition of pharmaceutically acceptable excipients, may be suspended or dissolved in an aqueous medium, with the resulting solution or suspension then being sterilized.

The chemistry described herein may also be used to prepare libraries of radiolabelled vectors suitable for screening as diagnostic drugs or in vivo imaging agents. Thus, a mixture of prosthetic groups of formula (II) or (IV) may be reacted with one or more compounds of formula (I) or (III) respectively using the methods described above to yield a library of radiolabelled vectors.

### EXAMPLES

The invention is illustrated by way of examples in which the following abbreviations are used:
HPLC: high performance liquid chromatography
DMF: N,N-dimethylformamide
DMSO: dimethylsulphoxide
ESI-MS: Electrospray Ionisation Mass Spectrometry
r.t.: room temperature
TOF-ESI-MS: time of flight electrospray ionisation mass spectrometry
FT-IR: Fourier transform infrared
ppm: parts per million
TFA: trifluoroacetic acid
ACN: acetonitrile

### Example 1

### Preparation of [¹⁸F]-4-(2-fluoroethyl)-triazol-1-yl-[KGFGK] using a copper loop reactor

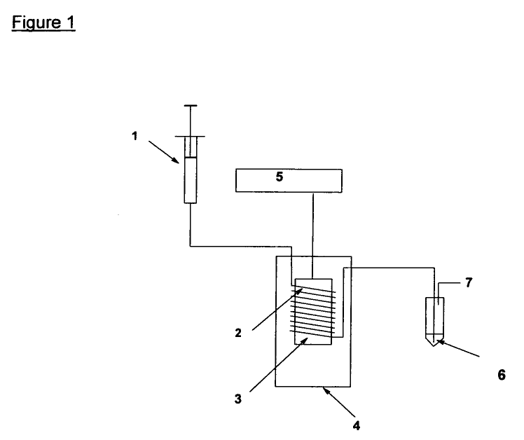

This Example is described with reference to FIG. 1. The heated copper tube has a length of 1.0 m and an inner diameter of 0.56 mm, tube volume 246 μl).

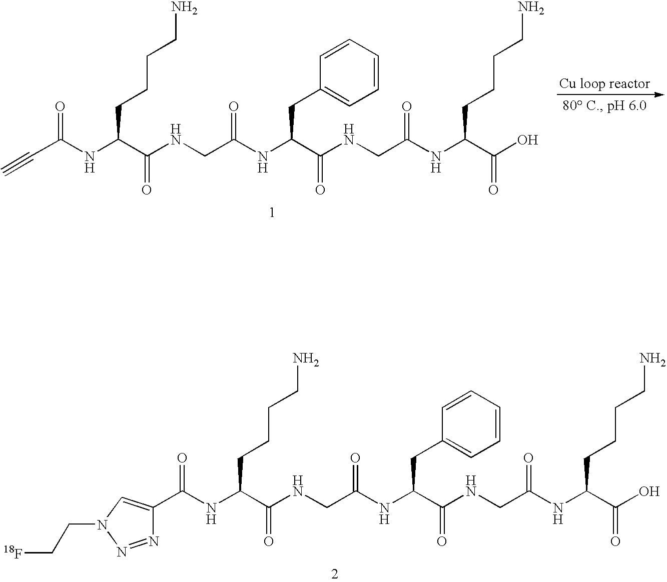

A solution of model peptide 1 (2.4 mg, 4.08 μmol), sodium phosphate buffer (0.2 ml, pH 6.0, 250 mM), DMF (0.05 ml) is mixed with [¹⁸F]2-fluoroethyl azide (0.6 mCi, 23 MBq) in acetonitrile (0.2 ml). A Hamilton Gastight glass syringe (1) is loaded with the labelling mixture which is subsequently pumped through a copper loop (2) at 80° C. with a flow rate of 0.2 ml/min. The electrical heating cylinder (3) can be heated up to 200° C. by a heating module (4) with a temperature control unit (5). The reaction mixture is trapped in a vial (6) fitted with a vent (7). The reaction mixture is analysed by HPLC, showing the formation of 2 with a radiochemical yield of 85% after 3-4 minutes. Re-injection of the labelling mixture into the copper loop reactor under identical conditions gives a radiochemical yield of >99%.

In Comparative Example 11, a lower labelling yield of 86% was achieved, although the peptide concentration was even higher in Comparative Example 11 (17 mM versus 9 Mm). Thus, this example demonstrates the benefits of using a copper loop reactor device for catalysing dipolar 1,3-cycloaddition reactions.

### Example 2

### Preparation of Compound 20 using Copper Loop Reactor

Preparation of compound 20 from compound 19 as referenced in Comparative Example 12.

Compound 19 (2.9 mg, 2.04 μmol) was dissolved in sodium phosphate buffer (100 μl, pH 6.0, 100 mM) with an additive of dimethylformamide (25 μl). After addition of compound II (518 μCi/19 MBq) in acetonitrile (100 μl), the mixture was pumped through the pre-heated copper loop reactor at 80° C. with 0.1 ml/min. Subsequently, the system was flushed with water (0.5 ml). HPLC analysis of the first and second fraction revealed a labelling efficiency of 9% and 34%, respectively. The total recovery of radioactivity from the system was 53%.

### Example 3

### Preparation of [¹⁸F]-4-(2-fluoroethyl)-triazol-1-yl-[KGFGK] using a copper loop reactor

A solution of model peptide 1 (2.4 mg, 4.08 μmol), sodium phosphate buffer (0.2 ml, pH 6.0, 250 mM), DMF (0.05 ml) is mixed with [¹⁸F]2-fluoroethyl azide (0.9 mCi, 34 MBq) in acetonitrile (0.2 ml). The mixture is pumped through the heated copper loop as described in example 1 but using a flow rate of 0.1 ml/min. The pass-through time of the mixture is 3 min and the total reaction time 10 min. Labelled peptide 2 is collected with 77% recovery (decay-corrected). The radiochemical purity is >99%. The copper loop reactor is cleaned using water (1 ml), water/TFA 1/1 (2 ml), water (2 ml), acetonitrile (3 ml), and drying using a stream of nitrogen (1 min, 50 ml/min). The experiment is repeated using the same starting activity of [¹⁸F]2-fluoroethyl azide. The radiochemical yield of isolated 2 is 71% (decay-corrected) and the radiochemical purity 98%.

### Preparation of Reference Compounds

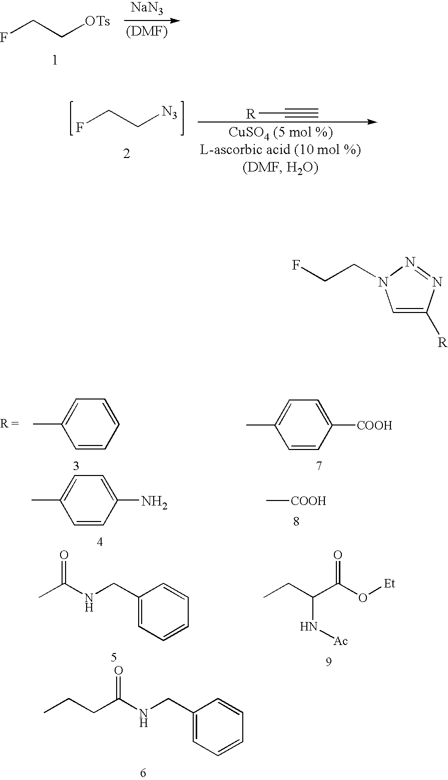

### Comparative Example 1

### Preparation of compound (2)—1-Azido 2-fluoroethane

Toluene-4-sulfonic acid 2-fluoro-ethyl ester, compound (1), was prepared as described by E. U. T. van Velzen et al. in Synthesis (1995) 989-997. Compound (1) (128 mg, 0.586 mmol) and sodium azide (114 mg, 1.758 mmol) were mixed with anhydrous DMF (10 ml) and stirred at room temperature for 48 hours. The reaction mixture was filtered, but product (2) was not isolated from the reaction solution.

### Comparative Example 2

### Preparation of compound (3)—1-(2-Fluoro-ethyl)-4-phenyl-1H-[1,2,3]triazole

Phenylacetylene (105 μl, 0.977 mmol) in DMF (1 ml) was added under nitrogen to a stirring solution of copper(II) sulphate pentahydrate (12 mg, 0.0489 mmol) and L-ascorbic acid (16 mg, 0.0977 mmol) in water (0.3 ml). After addition of compound (2) (1.172 mmol) in DMF (5 ml), stirring was continued at room temperature for 21 hours. The reaction mixture was diluted with water (5 ml), and the crude product was extracted with dichloromethane (3×5 ml) and washed with sodium bicarbonate solution (10%, 3×10 ml), and brine (1×5 ml). After drying over sodium sulphate, the solvent is removed under reduced pressure and the crude material purified using flash chromatography (silica, hexane/ethylacetate).

Yield: 32 mg (17%) white crystals, m.p. 83-85° C.

¹H-NMR (CDCl₃): δ=4.70 (m, 1H, CH₂), 4.76 (m, 1H, CH₂), 4.80 (m, 1H, CH₂), 4.89 (m, 1H, CH₂), 7.35 (tt, 1.0 Hz, 7.5 Hz, 1H, HAr), 7.44 (m, 2H, HAr), 7.84 (m, 2H, HAr), 7.89 (d, 1 Hz, 1H, CH-triazole) ppm

GC-MS: m/z=191

TOF-ESI-MS: found m/z=192.0935 [MH]⁺, calculated for C₁₀H₁₀N₃F [MH]⁺ m/z=192.0932

### Comparative Example 3

### Preparation of compound (4)—4-[1-(2-Fluoro-ethyl)-1H-[1,2,3]triazol-4-yl]′-phenylamine

4-Ethynylaniline (40 mg, 0.344 mmol) in DMF (0.7 ml) was added under nitrogen to a stirring solution of copper(II) sulphate pentahydrate (129 mg, 0.516 mmol) and L-ascorbic acid (182 mg, 1.032 mmol) in water (1.2 ml). After addition of compound (2) (0.287 mmol) in DMF (2.45 ml), stirring was continued at room temperature for 4 hours. The reaction mixture was quenched with sodium hydroxide solution (1 M, 5 ml). The product was extracted with ethyl acetate (3×5 ml), washed with water (5 ml), and brine (2 ml). After drying over sodium sulphate, the solvent was removed under reduced pressure and the crude material purified using flash chromatography (silica, hexane/ethylacetate). Yield: 15 mg (25%) beige crystals, m.p. 79-82° C.

¹H-NMR (CDCl₃): δ=4.70 (m, 1H, CH₂), 4.72 (m, 1H, CH₂), 4.77 (m, 1H, CH₂), 4.88 (m, 1H, CH₂), 6.74 (m, 2H, HAr), 7.63 (m, 2H, HAr), 7.74 (d, 0.1 Hz, 1H, CH-triazole) ppm

TOF-ESI-MS: found m/z=207.1030 [MH]⁺, calculated for C₁₀H₁₁N₄F [MH]⁺ m/z=207.1040

### Comparative Example 4

### Preparation of compound (5)—1-(2-Fluoro-ethyl)-1H-[1,2,3]triazole-4-carboxylic acid benzylamide

Propynoic acid benzylamide (50 mg, 0.314 mmol) that was prepared following the protocol of G. M. Coppola and R. E. Damon in Synthetic Communications 23 (1993) 2003-2010, was dissolved in DMF (1 ml) and added under nitrogen to a stirring solution of copper(II) sulphate pentahydrate (3.9 mg, 0.0157 mmol) and L-ascorbic acid (11 mg, 0.0628 mmol) in water (0.4 ml). After addition of compound (2) (0.377 mmol) in DMF (3.2 ml), stirring was continued at room temperature for 48 hours. The reaction mixture was diluted with sodium bicarbonate (10%, 5 ml), and the crude product was extracted with dichloromethane (3×5 ml) and washed with brine (5 ml). After drying over sodium sulphate, the solvent was removed under reduced pressure and the crude material purified by recrystallization from ethylacetate/diethylether.

Yield: 8 mg (10%) white crystals, m.p. 165-167° C.

¹H-NMR (CDCl₃): δ=4.70 (m, 6H, CH₂), 7.34 (m, 5H, HAr), 7.46 (m, 1H, NH), 8.20 (s, 1H, CH-triazole) ppm

TOF-ESI-MS: found m/z=249.1143 [MH]⁺, calc. for C₁₂H₁₃N₄₀F [MH]⁺ m/z=249.1146

### Comparative Example 5

### Preparation of compound (6)—N-Benzyl-3-[1-(2-fluoro-ethyl)-1H-[1,2,3]-triazol-4-yl]-propionamide

Pent-4-ynoic acid benzylamide—This compound was synthesised using a similar method as described by G. M. Coppola and R. E. Damon (see example 4) except with isolating of the N-succinimidyl intermediate.

Yield: 100 mg (53%) white needles, m.p. 50-55° C.

¹H-NMR (CDCl₃): δ=1.98 (m, 1H, alkyne-CH), 2.44 (m, 2H, CH₂), 2.56 (m, 2H,CH₂), 4.46 (d, 2H, CH₂N), 7.29-7.25 (m, 5H, HAr) ppm

FT-IR (film): 1651, 1629 cm⁻¹

TOF-ESI-MS: found m/z=188.1073 [MH]⁺, calc. for C₁₂H₁₃NO [MH]⁺ m/z=188.1070

N-Benzyl-341-(2-fluoro-ethyl)-1H-[1,2,3]-triazol-4-yl]-propionamide—Pent-4-ynoic acid benzylamide (50 mg, 0.267 mmol) in methanol (0.5 ml), compound (2) (0.320 mmol) in DMF (2.62 ml), and diisopropylamine (0.233 ml, 1.335 mmol) are added under nitrogen to a stirring suspension of copper(I) iodide (255 mg, 1.335 mmol) in methanol (0.8 ml). Stirring was continued at room temperature for 2 hours. The reaction mixture was quenched with a solution of sodium hydrogenphosphate (1 g) in water (10 ml) and filtered through Celite. The crude product was extracted with ethyl acetate (3×20 ml), and washed with brine (20 ml). After drying over sodium sulphate, the solvent was removed under reduced pressure and the crude material purified by column chromatography using silica and ethylacetate/hexane.

Yield: 19 mg (26%) white crystals, m.p. 127-133° C.

¹H-NMR (CDCl₃): δ=2.66 (t, 7.0 Hz, 2H, CH₂), 3.09 (t, 7.0 Hz, 2H, CH₂), 4.40 (d, 5.7 Hz, 2H, benzyl-CH₂), 4.56 (m, 2H, CH₂), 4.61 (m, 2H, CH₂), 4.70 (m, 2H, CH₂), 4.80 (m, 2H, CH₂), 6.0 (s, 1H, NH), 7.0-7.3 (m, 5H, HAr), 7.44 (s, 1H, CH-triazole) ppm

TOF-ESI-MS: found m/z=277.1474 [MH]⁺, calc. for C₁₂H₁₃N₄₀F [MH]⁺ m/z=277.1459

### Comparative Example 6

### Preparation of Compound (7)—4-[1-(2-Fluoro-Ethyl)-1H-[1,2,3]triazol-4-yl]-benzoic acid

Sodium 4-ethynylbenzoate (50 mg, 0.297 mmol) in DMF (1.5 ml) was added under nitrogen to a stirring solution of copper(II) sulphate pentahydrate (3.7 mg, 0.0149 mmol) and L-ascorbic acid (10.5 mg, 0.0595 mmol) in water (0.2 ml). After addition of compound (2) (0.356 mmol) in DMF (0.76 ml), stirring was continued at room temperature for 12 hours. The reaction mixture was diluted with HCl (20 ml, 1 M). The crude product was extracted with ethyl acetate (3×10 ml) and washed with brine (10 ml). After drying over sodium sulphate, the solvent was removed under reduced pressure ands the crude material recrystallized from ethylacetate/hexane.

Yield: 37 mg (52%) white crystals, m.p. 236-240° C.

¹H-NMR (DMSO-d₆): δ=4.74 (m, 1H, CH₂), 4.80 (m, 2H, CH₂), 4.90 (m, 1H, CH₂), 8.70 (s, 1 Hz, 1H, CH-triazole) ppm

TOF-ESI-MS: found m/z=236.0838 [MH]⁺, calc. for C₁₁H₁₀N₃O₂F [M]⁺ m/z=236.0830

### Comparative Example 7

### Preparation of compound (8)—1-(2-Fluoro-ethyl)-1H-[1,2,3]triazole-4-carboxylic acid

Propiolic acid (60 μl, 0.977 mmol) in DMF (0.5 ml) was added under nitrogen to a stirring solution of copper(II) sulphate pentahydrate (12 mg, 0.0489 mmol) and L-ascorbic acid (34 mg, 0.135 mmol) in water (0.4 ml). After addition of compound (2) (1.172 mmol) in DMF (2.5 ml), stirring was continued at room temperature for four hours. The reaction mixture was quenched with HCl (20 ml, 1 M), and the crude product was extracted with ethyl acetate (3×20 ml). After washing with brine (5 ml) and drying over sodium sulphate, the solvent was removed under reduced pressure and the product purified by recrystallisation from ethyl acetate/hexane.

Yield: 16 mg (10%) white crystals, m.p. 160-165° C.

¹H-NMR (DMSO-d₆): δ=4.74 (m, 1H, CH₂), 4.80 (m, 2H, CH₂), 4.90 (m, 1H, CH₂), 8.71 (s, 1H, CH-triazole) ppm

TOF-ESI-MS: found m/z=160.0518 [MH]⁺, calc. for C₅H₆N₃O₂F [MH]⁺ m/z=160.0517

### Comparative Example 8

### Preparation of compound (9)—2-Acetylamino-3-[1-(2-fluoro-ethyl)-1H-[1,2,3]triazol-4-yl]-propionic acid ethyl ester

2-Acetylamino-pent-4-ynoic acid ethyl ester (200 mg, 1.09 mmol) in methanol (1 ml) was added under nitrogen to copper powder (200 mg, 40 mesh), followed by a solution of compound (2) (1.09 mmol) in DMF (3 ml). The mixture was stirred for 90 minutes and then heated at 80° C. for three hours. Compound (9) was isolated by reverse phase flash chromatography (acetonitrile/water).

Yield: 145 mg (49%) oil, crystals upon storing at 4° C., m.p. 55-60° C.

¹H-NMR (CDCl₃): δ=1.13 (t, 3H, CH₂CH₃), 1.82 (s, 3H, CH₃), 2.97 (dd, ²J=14.9 Hz, ³J=8.5 Hz, 1H, propionic-CH₂), 3.07 (dd, ²J=14.9 Hz, ³J=6.0 Hz, 1H, propionic-CH₂), 4.05 (m, 2H, OCH₂CH₃), 4.47 (m, 1H, CH), 4.46 (m, 1H, CH₂), 4.64 (m, 1H, CH₂), 4.70 (m, 1H, CH₂), 4.81 (m, 1H, CH₂), 7.89 (s, 1H, triazole-CH), 8.31 (d, 1H, NH) ppm

TOF-ESI-MS: found m/z=273.1372 [MH]⁺, calc. for C₁₁H₁₇N₄O₃F [MH]⁺ m/z=273.1357

### Radiochemistry

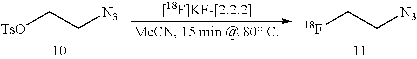

### Comparative Example 9

### Preparation of compound (11)—[¹⁸F]-Azido-2-fluoro-ethane

¹⁸F-Fluoride was produced by a cyclotron using the ¹⁸O(p,n)¹⁸F nuclear reaction with 19 MeV proton irradiation of an enriched [¹⁸O]H₂O target. After the irradiation, a mixture of Kryptofix® (5 mg), potassium carbonate (1 mg), and acetonitrile (1 ml) was added to ¹⁸F-water (1 ml). The solvent was removed by heating at 80° C. under a stream of nitrogen (100 ml/min). Afterwards, acetonitrile (0.5 ml) was added and evaporated under heating and nitrogen stream. This procedure was repeated twice. After cooling to room temperature, a solution of compound (10) [1.5 μl; prepared according to the method of Z. P. Demko and K. B. Sharpless, Org. Lett. 3 (2001) 4091] in anhydrous acetonitrile (0.2 ml) was added. The reaction mixture was stirred for 30 min at 80° C. Compound (11) was isolated with a decay-corrected radiochemical yield of 40±14% (n=7) through distillation [efficiency: 76±8% (n=7)].

### Comparative Example 10

### Preparation of compounds (12)-(16)—[¹⁸F][1-(2-Fluoro-ethyl)-1H-[1,2,3]triazoles

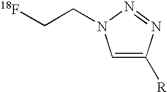

| Compound | R | R. C. Y.* |
|---|---|---|
| 12 | 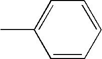 | 39%** |
| 13 | 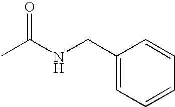 | 7% |
| 14 | 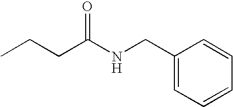 | <1% |
| 15 | 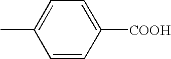 | 69% |
| 16 | 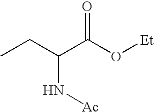 | >99% |

*by HPLC, **isolated, one-pot reaction

A solution of the alkyne reagent (0.015 mmol) in DMF (0.1 ml) was added to a mixture of copper(II) sulphate (5 equivalents) and L-ascorbic acid (20 equivalents) under nitrogen. A solution of compound (11) in acetonitrile (0.2 ml) was added. After stirring for 30 min at 80° C., the reaction mixture was analyzed by HPLC.

### Comparative Example 11

### Preparation of compound (18)—[¹⁸F](S)-6-Amino-2-(2-{(S)-2-[2-((S)-6-amino-2-{[4-(2-fluoro-ethyl)-[1,2,3]triazole-1-carbonyl]-amino}-hexanoylamino)-acetylamino]-3-phenyl-propionylamino}-acetylamino)-hexanoic acid

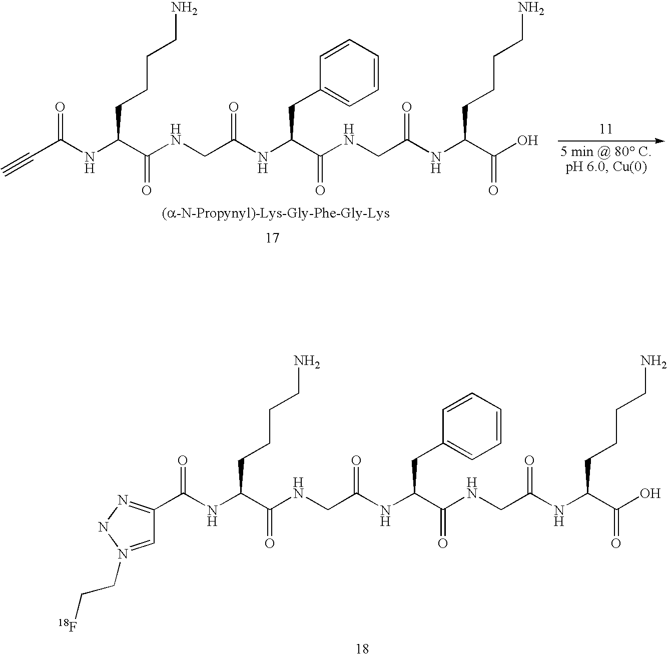

Compound (17) (1 mg, 1.7 μmol) was dissolved in sodium phosphate buffer (pH 6.0, 0.25 M, 0.05 ml). Compound (11) (175 μCi, 6.5 MBq) in acetonitrile (0.05 ml) was added followed by copper granules (400 mg, 10-40 mesh). The mixture was heated for 5 minutes at 80° C. HPLC analysis shows 86% of radiolabelled peptide (18).

### Comparative Example 12

### Preparation of compound (20)

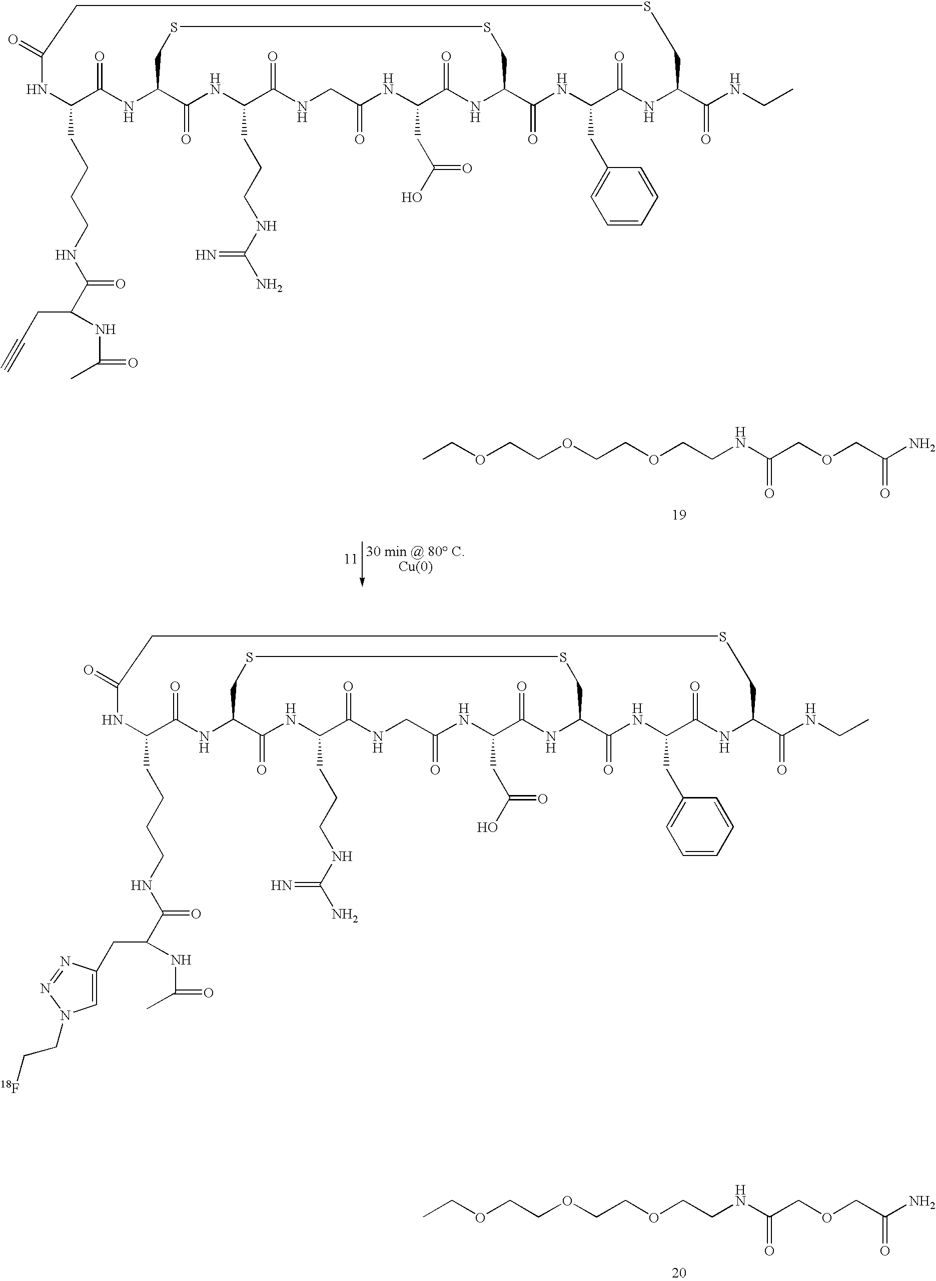

### (i) Preparation of compound 19: Cys2-6; c[CH₂CO-Lys(DL-Pra-Ac)-Cys-Arg-Gly-Asp-Cys-Phe-Cys]-CCX6-NH₂

Ac-DL-Pra-OH (31 mg), (7-Azabenzotriazole-1-yloxy)tripyrrolidinophosphonium hexafluorophosphate (PyAOP) (104 mg) and N-methylmorpholine (NMM) (88 NL) were dissolved in dimethylformamide (DMF) (3 mL) and the mixture stirred for 5 minutes prior to addition of ClCH₂CO-Lys-Cys(tBu)-Arg-Gly-Asp-Cys(tBu)-Phe-Cys-PEG-NH₂ (126 mg) prepared as described in WO2005/003166 dissolved in DMF (4 mL). The reaction mixture was stirred for 45 minutes. More ClCH₂CO-Lys-Cys(tBu)-Arg-Gly-Asp-Cys(tBu)-Phe-Cys-PEG-NH₂ (132 mg) and NMM (44 NL) were added and stirring continued for 45 minutes. DMF was then evaporated in vacuo, the residue (5 mL) diluted with 10% acetonitrile (ACN)/water (100 mL) and the product purified using preparative HPLC.

### Purification and Characterisation

Purification by preparative HPLC (gradient: 10-40% B over 60 min where A=H₂O/0.1 TFA and B=ACN/0.1% TFA, flow rate: 50 mL/min, column: Phenomenex Luna 5μ C18 (2) 250×50 mm, detection: UV 214 nm, product retention time: 31.3 min) of the diluted residue afforded 170 mg pure AH-112145.

The pure product was analysed by analytical HPLC (gradient: 10-40% B over 10 min where A=H₂O/0.1% TFA and B=ACN/0.1% TFA, flow rate: 0.3 mL/min, column: Phenomenex Luna 3μ C18 (2) 50×2 mm, detection: UV 214 nm, product retention time: 6.32 min). Further product characterisation was carried out using electrospray mass spectrometry (MH⁺ calculated: 1395.5, MH⁺ found: 1395.7).

### (ii) Preparation of Compound 20

Compound (19) (0.5 mg, 0.35 μmol) was dissolved in sodium phosphate buffer (pH 6.0, 50 mM) and mixed with a solution of compound (11) (25 μl, 728 μCi/25 MBq) and copper powder (200 mg, 40 mesh). After heating for 15 minutes at 70° C., the mixture is analysed by radio HPLC.

The conjugation product (20) was isolated using semipreparative HPLC (column Luna C18(2), 100×10 mm, flow rate 2.0 ml/min; solvent A: water (0.085% phosphoric acid v/v), solvent B: water (30° A) ethanol v/v), gradient: 50% B to 100% B in 15 minutes. The labelled peptide (20) was obtained with a decay-corrected radiochemical yield of 10% and a radiochemical purity of >99%. The identity of the radioactive product peak (k′=2.03) was confirmed by co-injection with a standard sample of compound (20).

### Comparative Example 13

### Optimization of Reaction Parameters for the Preparation of Compound (20)

General procedure: To a solution of compound (19) (0.5 mg, 0.35 μmol) in buffer (50 μl; buffer A: sodium phosphate, pH 6.0, 50 mM; buffer B: sodium carbonate, pH 9.3, 50 mM) is added compound (11) (0.1 mCi, 3.7 MBq) in acetonitrile (100 μl), followed by copper catalyst (catalyst 1: copper granules 10+40 mesh, catalyst 2: copper powder −40 mesh, catalyst 3: copper powder, dendritic, 3 μm). The mixture was incubated for 15 minutes at 80° C. and analyzed by HPLC.

TABLE 2
Labelling efficiency of compound (19) to form compound (20)
depending on pH and catalyst (400 mg) as measured by HPLC

| Buffer | Catalyst 1 | Catalyst 2 | Catalyst 3 |
|---|---|---|---|
| A | 12% | 44% | —* |
| B | — | 33% | —* |

*no UV peak for peptide precursor found

TABLE 3
Labelling efficiency of compound (19) to form compound (20)
depending amount of catalyst 3 at pH 6.0 (buffer A).

| Amount of catalyst 3 | Labelling efficiency of compound (20) |
|---|---|
| 200 mg | 23% |
| 100 mg | 37% |
| 50 mg | 27% |

The invention described and claimed herein is not to be limited in scope by the specific embodiments herein disclosed, since these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

1. A method for labelling a vector comprising reaction of a compound of formula (I) with a compound of formula (II): 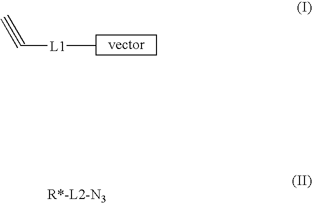
or,
a compound of formula (III) with a compound of formula (IV) 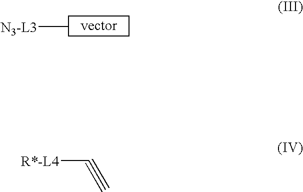
wherein:
L1, L2, L3, and L4 are each Linker groups;
R* is a reporter moiety;
to give a conjugate of formula (V) or (VI) respectively: 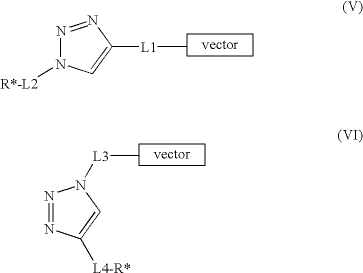
wherein L1, L2, L3, L4, and R* are as defined above;
characterised in that the reaction is performed in a narrow bore copper vessel.

2. A method according to claim 1 wherein R* comprises a radionuclide.

3. A method according to claim 1 wherein R* comprises a positron-emitting radionuclide, preferably ¹¹C or ¹⁸F.

4. A method according to claim 1 wherein the vector is a peptide, protein, hormone, cell, bacterium, virus, or small drug-like molecule, most suitably a peptide.

5. A method according to claim 1 wherein the vector is Arg-Gly-Asp peptide or an analogue thereof.

6. A method according to claim 1 wherein the vector is a peptide comprising the fragment:

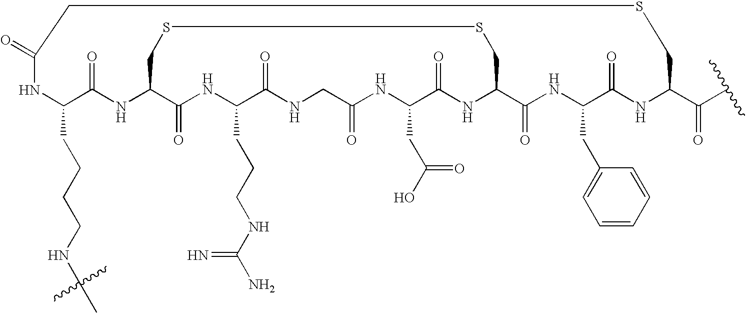

7. A method according to claim 1 wherein the vector is the peptide of formula (A): 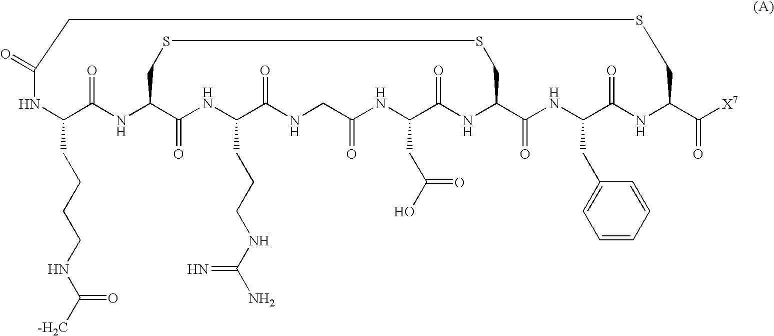
wherein X⁷ is either —NH₂ or 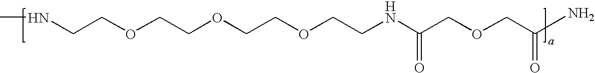
wherein a is an integer of from 1 to 10, preferably a is 1.

8. Microfluidic device for performing a method according to claim 1 characterised in that said device comprises a device body defining a first apperture, a second apperture, and at least one elongate microfluidic passageway in fluid communication therebetween wherein at least a portion of the microfluidic passageway is defined by a metallic copper portion of the device body.

9. Microfluidic device according to claim 8 wherein the device body defining the at least one microfluidic passageway is formed from metallic copper.

10. Microfluidic device according to claim 8 wherein the device body further comprises a base portion and a cover portion defining said first apperture, second apperture, and at least one elongate microfluidic passageway in fluid communication therebetween.

11. Microfluidic device according to claim 10 wherein the microfluidic passageway comprises a channel formed in either the base portion or the cover portion or in both thereof in overlying registry.

12. Microfluidic device for performing a method according to claim 1 characterised in that said device comprises a device body defining a first apperture, a second apperture, and at least one elongate microfluidic passageway in fluid communication therebetween wherein at least a portion of the microfluidic passageway is defined by a metallic copper portion of the device body wherein the microfluidic passageway contains a compound of formula (I), (II), (III), or (IV) as defined in any of claim 1.
